(19) United States
(12) Patent Application Publication
(10) Pub. No.: US 2026/0335145 A1
(43) Pub. Date: Oct. 29, 2026

(54) MULTI-LAYER TRANSFER TAPES FOR MEDICAL ARTICLES

(71) Applicant: Solventum Intellectual Properties Company, Maplewood, MN (US)

(72) Inventors: Hironobu ISHIWATARI, Saint Paul, MN (US); Christoph SCHUELL, Saint Paul, MN (US); Kiu-Yuen TSE, Saint Paul, MN (US)

(73) Assignee: Solventum Intellectual Properties Company, Maplewood, MN (US)

(21) Appl. No.: 19/115,615

(22) PCT Filed: Mar. 10, 2023
(86) PCT No.: PCT/IB2023/059934

§ 371 Date: Mar. 26, 2025

Related U.S. Application Data
(60) Provisional application No. 63/576,438, filed on Oct. 10, 2022.

(51) Int. Cl. A61F 13/02; A61L 15/24; C09J 7/21; C09J 7/38; C09J 7/40

(52) U.S. Cl. CPC A61F 13/0289; A61F 13/0266; A61L 15/24; C09J 7/21; C09J 7/385; C09J 7/403; C09J 2433/00

(57) ABSTRACT

Transfer tapes useful in preparing medical articles include a release liner, a (meth)acrylate-based pressure sensitive adhesive layer disposed on and in contact with the release liner, and a radiation-cured siloxane gel adhesive layer disposed on and in contact with the (meth)acrylate-based pressure sensitive adhesive layer. The siloxane gel adhesive layer is formed by radiation curing of a non-functional siloxane fluid when the fluid is in contact with the (meth)acrylate-based layer. The transfer tape has high interfacial adhesion between the (meth)acrylate-based layer and the siloxane gel adhesive layer such that the siloxane layer cannot be peeled from the (meth)acrylate layer.

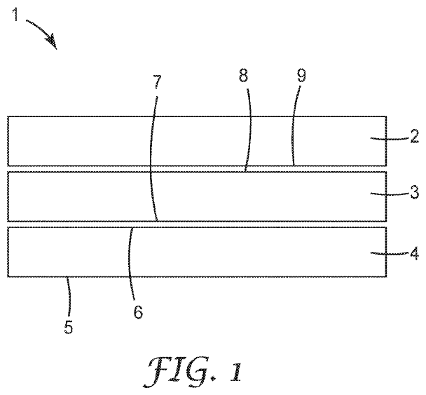

### SUMMARY

Disclosed herein are multi-layer transfer tapes, articles prepared with these transfer tapes, and methods for preparing articles with multi-layer transfer tapes. In some embodiments, the transfer tape comprises a release liner with a first major surface and a second major surface, wherein at least the second major surface is a release surface, a (meth)acrylate-based pressure sensitive adhesive layer with a first major surface and a second major surface, where the first major surface is in contact with the second major surface of the release liner, and a radiation-cured siloxane gel adhesive layer with a first major surface and a second major surface wherein the first major surface of the siloxane gel adhesive layer is in contact with the second major surface of the (meth)acrylate-based pressure sensitive adhesive layer. The radiation-cured siloxane gel adhesive layer is formed by radiation curing of a non-functional siloxane fluid, where the non-functional siloxane fluid is in contact with the (meth)acrylate-based pressure sensitive adhesive layer when cured such that the interfacial adhesion of the (meth)acrylate-based pressure sensitive adhesive and the radiation-cured siloxane gel adhesive layer is sufficiently strong that the radiation-cured siloxane gel adhesive layer is not peelable from the (meth)acrylate-based pressure sensitive adhesive layer. In many embodiments, the radiation curing comprises E-beam curing.

Also disclosed are methods for preparing articles, particularly medical articles. In some embodiments, the method comprises forming a transfer tape on a release liner, where the transfer tape is described above. These transfer tape articles can be removed from the release liner and laminated to a wide range of substrates to form other articles. Examples of the articles that can be formed from the transfer tape articles include a wound dressing, a medical drape, a medical bandage, a wound cover, a negative pressure wound therapy article, or a wearable medical construction.

### BRIEF DESCRIPTION OF THE DRAWINGS

The present application may be more completely understood in consideration of the following detailed description of various embodiments of the disclosure in connection with the accompanying drawing.

The FIGURE is a cross sectional view of an article of this disclosure.

### DETAILED DESCRIPTION

The widespread use of adhesives in medical applications has led to the development of adhesives and adhesive articles that are gentle to the skin. Some of these adhesives are pressure sensitive adhesives and others are gel adhesives. The application of pressure sensitive adhesives, including silicone pressure sensitive adhesives, for adhering to skin is known in the art and many examples are commercially available. However, some pressure sensitive adhesives have limited application for adhesion to skin. For instance, skin damage may result during the removal of a pressure sensitive adhesive that exhibits surface adhesion to skin that is too high. Alternatively, if the surface adhesion to skin is reduced, the pressure sensitive adhesive may lack sufficient holding power to be useful or will lose the room temperature tackiness that makes easy application of the adhesive possible.

Another class of adhesives used in medical applications are silicone gels. As used herein, the terms “siloxane” and “silicone” are used interchangeably. The term siloxane is replacing silicone in common usage, but both terms are used in the art. Silicone gel (crosslinked poly dimethylsiloxane (“PDMS”)) materials have been used for dielectric fillers, vibration dampers, and medical therapies for promoting scar tissue healing. Lightly crosslinked silicone gels are soft, tacky, elastic materials that comprise relatively high levels of fluids (liquids). Silicone gels are typically softer than silicone pressure sensitive adhesives, resulting in less discomfort when adhered to skin. The combination of reasonable adhesive holding power on skin and low skin trauma upon removal, make silicone gels suitable for gentle to skin adhesive applications.

Silicone gel adhesives provide good adhesion to skin with gentle removal force and have the ability to be repositioned. Examples of commercially available silicone gel adhesive systems include products marketed with the trade names: Dow Corning MG 7-9850, WACKER 2130, BLUESTAR 4317 and 4320, and NUSIL 6345 and 6350. These gentle to the skin adhesives are formed by an addition cure reaction between vinyl-terminated PDMS and hydrogen terminated PDMS, in the presence of a hydrosilylation catalyst (e.g., platinum complex). Vinyl-terminated and hydrogen terminated PDMS chains are referred to as ‘functionalized’ silicones due to their specific chemical moieties. Individually, such functional silicones are generally not reactive; however, together they form a reactive silicone system. Additionally, silicone resins (tackifiers sometimes referred to as “silicate resins”) and PDMS with multiple hydrogen functionalities (crosslinkers) can be formulated to modify the adhesive properties of the gel.

There are downsides to the use of these types of materials. For example, they required the use of specialized “functionalized” silicone materials. Also, they typically required the use of a catalyst, often a metal-containing catalyst such as platinum or palladium catalysts. These catalysts are expensive and leave metal-containing residues in the cured compositions. An alternative to the catalyst-promoted curing of such silicone materials is the use of free radical polymerization to cure or crosslink the silicone pressure sensitive adhesive or gel formulations. These polymerizations require initiation by a free radical source, such as, for example, the high temperature degradation of organic peroxides. This curing technique can be undesirable due to the acidic residues left in the film from the curing chemistry, which are corrosive and unsuitable for skin contact.

Recently siloxane-based gel adhesives and sealants have been prepared that cure and crosslink at room temperature without generating undesirable catalyst or initiator residues and do not require specialized functionalized starting materials, rather they are prepared either from silanol-functional siloxane materials or siloxane materials without any reactive functional groups. These siloxane-based gel compositions can be formed by a condensation reaction in the case of silanol-functional materials, or by the generation of free radicals by exposure to an electron beam (e-beam) or gamma radiation in the case of siloxane materials without any reactive functional groups. In the condensation reaction, two silanol groups (that is to say, terminal —SiOH groups) condense to form —Si—O—Si— linkages and a molecule of water (H₂O).

These siloxane-based gel adhesives and sealants have excellent wetting and flow characteristics, due to the very low glass transition temperature (Tg) and modulus of the polysiloxane matrix and achieve their adhesive holding power on the rough skin surface due to mechanical interlock and energy dissipation within the gel adhesive. Additionally, the low surface adhesion of silicone gels prevents the adhesive from tightly attaching to hair or skin cells during skin wear, further reducing the instance of pain during removal. This results in minimal to no skin trauma upon removal.

Thus, the use of pressure sensitive adhesives in medical applications have issues because they can have good adhesion to a wide variety of substrates (for example, to skin as well as to tubing, drapes, tape backings, and the like) but they can cause skin damage. Gel adhesives on the other hand can have desired low skin trauma, but these adhesives also have low adhesion, both to skin and to other substrates such as tubing, drapes, tape backings, and the like. Thus, the need remains for adhesives suitable for medical uses that have high adhesion to a wide range of substrates without causing skin damage.

In this disclosure dual-sided adhesives are described that have one adhesive surface that has desirable adhesion to mammalian skin and another adhesive surface that has desirable adhesion to a wide range of substrates.

Dual-sided tapes, also called “transfer tapes” are adhesive tapes that have adhesive on both exposed surfaces. In some transfer tapes, the exposed surfaces are simply the two surfaces of a single adhesive layer. Other transfer tapes are multi-layer transfer tapes with at least two adhesive layers that may be the same or different, and in some instances intervening layers that may not be adhesive layers. For example, a multi-layer transfer tape may be a 3-layer construction with an adhesive layer, a film layer and another adhesive layer. The film layer can provide handling and/or tear strength or other desirable properties. Many transfer tapes have two different adhesive layers that have been laminated together without a film layer between. Generally, transfer tapes are supplied disposed on a release liner. This permits the transfer tape, with two exposed adhesive surfaces, to be handled, transported without contacting the adhesive surfaces. In some instances, the transfer tape is supplied with the transfer tape disposed between two release surfaces. This can be achieved by the use of two release liners or by using a double-sided release liner and the transfer tape is supplied as a roll. In a roll, one adhesive surface is disposed on one surface of the double-sided release liner and upon rolling up the transfer tape the second surface of the double-sided release contacts the second adhesive surface of the transfer tape.

Having two different types of adhesive layers in the transfer tape provides a variety of advantages as well as a variety of challenges. Among the advantages of transfer tapes having two different types of adhesive layers is the ability to bond to different types of surfaces, including very different types of surfaces, with a single transfer tape.

Among the challenges of having two different types of adhesive layers in the transfer tape can include the potential for a weak boundary layer between the two adhesive layers. By this it is meant that because the two adhesive layers are different, they may not bond well to each other creating a weak bond between the two adhesive layers (often called a “weak boundary layer”). This weak boundary layer can be problematic in that it can become a locus of failure when the transfer tape is used to form adhesive bonds.

In this disclosure, multilayer transfer tapes are presented which have two different adhesive layers with no intervening layers. The transfer tapes have one adhesive surface that is a siloxane-based adhesive layer, typically a siloxane gel adhesive, that is suitable for adhesion to mammalian skin. The other adhesive layer is a pressure sensitive adhesive layer that is suitable for bonding to a wide range of substrates and articles, including for example: tape backings to generate a tape article, a medical device to prepare a wearable adhesive article; or other film substrates to prepare a wide range of medical articles.

The term “adhesive” as used herein refers to polymeric compositions useful to adhere together two adherends. Examples of adhesives are pressure sensitive adhesives and gel adhesives.

Pressure sensitive adhesive compositions are well known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as pressure sensitive adhesives are polymers designed and formulated to exhibit the requisite viscoelastic properties resulting in a desired balance of tack, peel adhesion, and shear holding power. Obtaining the proper balance of properties is not a simple process.

As used herein, the term “gel adhesive” refers to a tacky semi-solid crosslinked matrix containing a liquid or a fluid that is capable of adhering to one or more substrates. The gel adhesives may have some properties in common with pressure sensitive adhesives, but they are not pressure sensitive adhesives. “Hydrogel adhesives” are gel adhesives that have water as the fluid contained within the crosslinked matrix.

The term “(meth)acrylate” refers to monomeric acrylic or methacrylic esters of alcohols. Acrylate and methacrylate monomers or oligomers are referred to collectively herein as “(meth)acrylates”. Materials referred to as “(meth)acrylate functional” are materials that contain one or more (meth)acrylate groups. Materials referred to as “(meth)acrylate-based” contain a majority or at least one (meth)acrylate monomer and may contain other co-polymerizable monomers.

The terms “siloxane-based” as used herein refer to polymers or units of polymers that contain siloxane units. The terms silicone or siloxane are used interchangeably and refer to units with dialkyl or diaryl siloxane (—SiR₂O—) repeating units.

The terms “room temperature” and “ambient temperature” are used interchangeably to mean temperatures in the range of 20 °C to 25 °C.

The terms “Tg” and “glass transition temperature” are used interchangeably. If measured, Tg values are determined by Differential Scanning calorimetry (DSC) at a scan rate of 10 °C/minute, unless otherwise indicated. Typically, Tg values for copolymers are not measured but are calculated using the well-known Fox Equation, using the homopolymer Tg values provided by the monomer supplier, as is understood by one of skill in the art.

The terms “polymer” and “macromolecule” are used herein consistent with their common usage in chemistry. Polymers and macromolecules are composed of many repeated subunits. As used herein, the term “macromolecule” is used to describe a group attached to a monomer that has multiple repeating units. The term “polymer” is used to describe the resultant material formed from a polymerization reaction.

The term “alkyl” refers to a monovalent group that is a radical of an alkane, which is a saturated hydrocarbon. The alkyl can be linear, branched, cyclic, or combinations thereof and typically has 1 to 20 carbon atoms. In some embodiments, the alkyl group contains 1 to 18, 1 to 12, 1 to 10, 1 to 8, 1 to 6, or 1 to 4 carbon atoms. Examples of alkyl groups include, but are not limited to, methyl, ethyl, n-propyl, isopropyl, n-butyl, isobutyl, tert-butyl, n-pentyl, n-hexyl, cyclohexyl, n-heptyl, n-octyl, and ethylhexyl.

The term “aryl” refers to a monovalent group that is aromatic and carbocyclic. The aryl can have one to five rings that are connected to or fused to the aromatic ring. The other ring structures can be aromatic, non-aromatic, or combinations thereof. Examples of aryl groups include, but are not limited to, phenyl, biphenyl, terphenyl, anthryl, naphthyl, acenaphthyl, anthraquinonyl, phenanthryl, anthracenyl, pyrenyl, perylenyl, and fluorenyl.

The terms “free radically polymerizable” and “ethylenically unsaturated” are used interchangeably and refer to a reactive group which contains a carbon-carbon double bond which is able to be polymerized via a free radical polymerization mechanism.

As was mentioned above, the current disclosure comprises transfer tapes that comprise two adhesive layers that are in contact with each other. The transfer tape comprises a (meth)acrylate-based pressure sensitive adhesive layer and a siloxane gel adhesive layer. The two adhesive layers, despite being very different in composition, adhere strongly to each other. This phenomenon is described as “interfacial adhesion”. In transfer tapes, weak interfacial adhesion can be an issue because the two adhesive layers can adhere more strongly to substrates than they adhere to each other. In this way, when the adhesive layer is removed, instead of the adhesive peeling away from the substrate surface, one of the adhesive layers peels away from the other adhesive layer.

A variety of techniques have employed in transfer tapes to overcome problems with weak interfacial adhesion. One way is to provide an interlayer that both adhesives adhere strongly to so that the interlayer provides a tie layer to hold the transfer tape together. The use of an interlayer is not always suitable, particularly in medical articles, since for example medical adhesive layers often desirably provide moisture vapor transfer. Other techniques to increase interfacial adhesion between adhesive layers of a transfer tape include using a primer layer or tie layer to increase the adhesion between the layers.

In the current disclosure, the development of strong interlayer adhesion is achieved by curing a layer of non-functional siloxane fluid, where the fluid is in contact with a (meth)acrylate-based pressure sensitive adhesive layer.

Disclosed herein are transfer tapes. The transfer tapes comprise a release liner with a first major surface and a second major surface, where at least the second major surface is a release surface, a (meth)acrylate-based pressure sensitive adhesive layer with a first major surface and a second major surface, wherein the first major surface is in contact with the second major surface of the release liner, and a radiation-cured siloxane gel adhesive layer with a first major surface and a second major surface wherein the first major surface of the siloxane gel adhesive layer is in contact with the second major surface of the (meth)acrylate-based pressure sensitive adhesive layer. The radiation-cured siloxane gel adhesive layer is formed by radiation curing of a non-functional siloxane fluid layer that is in contact with the (meth)acrylate-based pressure sensitive adhesive layer. The interfacial adhesion of the (meth)acrylate-based pressure sensitive adhesive and the radiation-cured siloxane gel adhesive layer is sufficiently strong that the radiation-cured siloxane gel adhesive layer is not peelable from the (meth)acrylate-based pressure sensitive adhesive layer.

A variety of types of radiation curing may be used to cure the non-functional siloxane fluid that is in contact with the (meth)acrylate-based pressure sensitive adhesive layer. Typically, the radiation curing is carried out with electron beam (E-beam), gamma radiation, or X-rays. In many embodiments, E-beam radiation is used.

As mentioned above, the transfer tapes comprise a release liner, a (meth)acrylate-based pressure sensitive adhesive layer disposed on the release liner, and a radiation-cured siloxane gel adhesive layer disposed on the (meth)acrylate pressure sensitive adhesive layer, where the siloxane gel adhesive layer is formed by radiation-curing of a layer of non-functionalized siloxane fluid. Each of these components is described in detail below.

The transfer tape comprises a release liner that serves as a carrier layer to allow manipulation of and protection for the transfer tape. A wide variety of release liners are suitable. Release liners are commonly used and well understood in the adhesive arts. Exemplary release liners include those prepared from paper (e.g., Kraft paper) or polymeric material (e.g., polyolefins such as polyethylene or polypropylene, ethylene vinyl acetate, polyurethanes, polyesters such as polyethylene terephthalate, and the like, and combinations thereof). At least some release liners are coated with a layer of a release agent such as a silicone-containing material or a fluorocarbon-containing material. Exemplary release liners include, but are not limited to, liners commercially available under the trade name SILFLU from Siliconature (Italy) that have a fluorosilicone release coating on polyethylene terephthalate film.

The transfer tape also comprises a layer of a (meth)acrylate-based pressure sensitive adhesive disposed on a releasing surface of the release liner. A wide range of (meth)acrylate-based pressure sensitive adhesives are suitable for the (meth)acrylate-based pressure sensitive adhesive layer of the transfer tape. These adhesives can be formed and delivered to form a layer in a wide variety of ways including solvent-based and solventless techniques. Among the solventless techniques are hot melt processing techniques. Particularly suitable (meth)acrylate-based pressure sensitive adhesives include copolymers derived from: (A) at least one monoethylenically unsaturated alkyl (meth)acrylate monomer (i.e., alkyl acrylate and alkyl methacrylate monomer); and (B) at least one monoethylenically unsaturated free-radically copolymerizable reinforcing monomer. The reinforcing monomer has a homopolymer glass transition temperature (Tg) higher than that of the alkyl (meth)acrylate monomer and is one that increases the glass transition temperature and cohesive strength of the resultant copolymer. Herein, “copolymer” refers to polymers containing two or more different monomers, including terpolymers, tetrapolymers, etc.

Monomer A, which is a monoethylenically unsaturated alkyl acrylate or methacrylate (i.e., (meth)acrylic acid ester), contributes to the flexibility and tack of the copolymer. Generally, monomer A has a homopolymer Tg of no greater than about 0 °C. Typically, the alkyl group of the (meth)acrylate has an average of about 4 to about 20 carbon atoms, or an average of about 4 to about 14 carbon atoms. The alkyl group can optionally contain oxygen atoms in the chain thereby forming ethers or alkoxy ethers, for example. Examples of monomer A include, but are not limited to, 2-methylbutyl acrylate, isooctyl acrylate, lauryl acrylate, 4-methyl-2-pentyl acrylate, isoamyl acrylate, sec-butyl acrylate, n-butyl acrylate, n-hexyl acrylate, 2-ethylhexyl acrylate, n-octyl acrylate, n-decyl acrylate, isodecyl acrylate, isodecyl methacrylate, and isononyl acrylate. Other examples include, but are not limited to, poly-ethoxylated or -propoxylated methoxy(meth)acrylates such as acrylates of CARBOWAX (commercially available from Union Carbide) and NK ester AM90G (commercially available from Shin Nakamura Chemical, Ltd., Japan). Suitable monoethylenically unsaturated (meth)acrylates that can be used as monomer A include isooctyl acrylate, 2-ethyl-hexyl acrylate, and n-butyl acrylate. Combinations of various monomers categorized as an A monomer can be used to make the copolymer.

Monomer B, which is a monoethylenically unsaturated free-radically copolymerizable reinforcing monomer, increases the glass transition temperature and cohesive strength of the copolymer. Generally, monomer B has a homopolymer Tg of at least about 10 °C. Typically, monomer B is a reinforcing (meth)acrylic monomer, including an acrylic acid, a methacrylic acid, an acrylamide, or a (meth)acrylate. Examples of monomer B include, but are not limited to, acrylamides, such as acrylamide, methacrylamide, N-methyl acrylamide, N-ethyl acrylamide, N-hydroxyethyl acrylamide, diacetone acrylamide, N,N-dimethyl acrylamide, N,N-diethyl acrylamide, N-ethyl-N-aminoethyl acrylamide, N-ethyl-N-hydroxyethyl acrylamide, N,N-dihydroxyethyl acrylamide, t-butyl acrylamide, N,N-dimethylaminoethyl acrylamide, and N-octyl acrylamide. Other examples of monomer B include itaconic acid, crotonic acid, maleic acid, fumaric acid, 2,2-(diethoxy)ethyl acrylate, 2-hydroxyethyl acrylate or methacrylate, 3-hydroxypropyl acrylate or methacrylate, methyl methacrylate, isobornyl acrylate, 2-(phenoxy)ethyl acrylate or methacrylate, biphenylyl acrylate, t-butylphenyl acrylate, cyclohexyl acrylate, dimethyladamantyl acrylate, 2-naphthyl acrylate, phenyl acrylate, N-vinyl formamide, N-vinyl acetamide, N-vinyl pyrrolidone, and N-vinyl caprolactam. Particularly suitable reinforcing acrylic monomers that can be used as monomer B include acrylic acid and acrylamide. Combinations of various reinforcing monoethylenically unsaturated monomers categorized as a B monomer can be used to make the copolymer.

Generally, the (meth)acrylate copolymer is formulated to have a resultant Tg of less than about 0 °C and more typically, less than about −10 °C. Such (meth)acrylate copolymers generally include about 60 parts to about 98 parts per hundred of at least one monomer A and about 2 parts to about 40 parts per hundred of at least one monomer B. In some embodiments, the (meth)acrylate copolymers have about 85 parts to about 98 parts per hundred or at least one monomer A and about 2 parts to about 15 parts of at least one monomer B.

The (meth)acrylate-based pressure sensitive adhesive layer can have a wide range of thicknesses. Typically, the (meth)acrylate-based pressure sensitive adhesive layer has a thickness of from 10-100 micrometers.

The (meth)acrylate-based pressure sensitive adhesive layer may be a continuous or a discontinuous layer. In many embodiments, the (meth)acrylate-based pressure sensitive adhesive layer is a continuous layer.

The (meth)acrylate-based pressure sensitive adhesive layer may be formed and disposed on the release liner, or a precursor mixture can be disposed on the release liner and then formed into the pressure sensitive adhesive. The precursor mixture can contain the monomer components described above together with an initiator. The precursor mixture can then be formed into the pressure sensitive adhesive layer by curing. If pre-formed, the pressure sensitive adhesive can be disposed on the release liner by coating techniques such as solvent coating, hot melt coating, and the like and the pressure sensitive adhesive layer can then be formed by drying or cooling.

The transfer tape also comprises a siloxane gel adhesive layer disposed on the (meth)acrylate-based pressure sensitive adhesive layer. This siloxane gel adhesive layer comprises a crosslinked siloxane matrix and siloxane fluid. The siloxane gel adhesive layer is formed by the radiation curing of a non-functional siloxane fluid. As used herein, “nonfunctional fluids” are ones that do not contain functional groups that can participate in the crosslinking reaction to form the siloxane matrix of the siloxane gel adhesive. This definition is the same as defined in US Patent Publication No. 2011/0212325 (Determan et al.), which describes the formation of siloxane gel adhesive from non-functional siloxane fluids.

A wide variety of non-functional siloxane fluids are suitable to form the crosslinked siloxane matrices of this disclosure. Generally, the siloxane fluids are described by Formula 1 below:

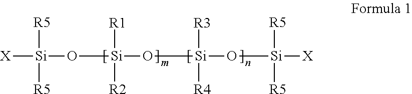

wherein R1, R2, R3, and R4 are independently selected from the group consisting of an alkyl group or an aryl group, each R5 is an alkyl group, each X is a non-functional group, and n and m are integers, and at least one of m or n is not zero. In some embodiments, one or more of the alkyl or aryl groups may contain a halogen substituent, e.g., fluorine. For example, in some embodiments, one or more of the alkyl groups may be —CH₂CH₂C₄F₉.

In the non-functional siloxane fluids of this disclosure, the X group in Formula 1 is a hydroxyl group or an R5 group. When X is a hydroxyl group, the fluid is a hydroxyl-functional polysiloxane, often called a “silanol-terminated siloxane fluid” or a “silanol fluid”. The X groups are frequently referred to as “terminal” groups and the R1, R2, R3, and R4 groups are referred to as “pendant” groups.

In some embodiments, R1 and R2 are alkyl groups and n is zero, i.e., the material is a poly(dialkylsiloxane). In some embodiments, the alkyl group is a methyl group, i.e., poly(dimethylsiloxane) (“PDMS”). In some embodiments, R1 is an alkyl group, R2 is an aryl group, and n is zero, i.e., the material is a poly(alkylarylsiloxane). In some embodiments, R1 is methyl group and R2 is a phenyl group, i.e., the material is poly(methylphenylsiloxane). In some embodiments, R1 and R2 are alkyl groups and R3 and R4 are aryl groups, i.e., the material is a poly(dialkyldiarylsiloxane). In some embodiments, R1 and R2 are methyl groups, and R3 and R4 are phenyl groups, i.e., the material is poly(dimethyldiphenylsiloxane).

In some commercially available embodiments, R1, R2, R3, R4, and R5 are all methyl groups, making the material a poly(dimethylsiloxane) or PDMS material. In other embodiments, at least some of the R1, R2, R3, and R4 are aryl groups.

Many suitable silanol-terminated siloxane fluids are commercially available. Numerous examples of materials are commercially available from, for example, Gelest, Inc. Morrisville, PA, Dow Corning, Midland MI, and Wacker Chemie AG, Munich, Germany. Particularly suitable examples include the silanol terminated PDMS (polydimethyl siloxane), commercially available as XIAMETER OHX-4070, from Dow Corning, Midland, MI, and the hydroxyl functional PDMS commercially available as AK1000000 from Wacker Chemie AG, Munich, Germany.

As mentioned above, the layer of non-functional siloxane fluid is radiation cured to form the siloxane gel adhesive layer. The non-functional siloxane fluid layer, in contact with (meth)acrylate-based pressure sensitive adhesive layer, is exposed to radiation to carry out the curing of the non-functional siloxane fluid layer. Typically, the radiation is E-beam radiation, although other types of radiation can also be used (such as gamma radiation or X-rays).

There are many advantages to this method of forming a siloxane gel adhesive layer. Because a non-functionalized siloxane fluid is disposed onto the (meth)acrylate-based pressure sensitive adhesive layer, and then crosslinked, the siloxane fluid that does not crosslink to form a matrix remains in the matrix as the fluid component of the gel adhesive. Also, any additional additives, such as siloxane tackifying resins, can be added to the non-functional siloxane fluid prior to disposal on the (meth)acrylate-based pressure sensitive adhesive layer, and thus is distributed throughout the mixture prior to the crosslinking. This not only provides for effective dispersal throughout the precursor mixture, it also much simpler to add the additives to the fluid than to the crosslinked gel.

The radiation-cured siloxane gel adhesive layer can have a wide range of thicknesses. In some embodiments, the radiation-cured siloxane gel adhesive layer has a thickness of from 25-500 micrometers.

As with the (meth)acrylate-based pressure sensitive adhesive layer, the radiation-cured siloxane gel adhesive layer can be continuous or discontinuous. In some particularly suitable embodiments, the (meth)acrylate-based pressure sensitive adhesive layer is a continuous layer and the radiation-cured siloxane gel adhesive layer is discontinuous. These embodiments can be prepared by forming a discontinuous layer of non-functional siloxane fluid on the (meth)acrylate-based pressure sensitive adhesive layer and then crosslinking. A discontinuous layer of non-functional siloxane fluid can be formed in a variety of ways. In some embodiments, the discontinuous layer of non-functional siloxane fluid is formed by printing to generate an adhesive pattern.

Also disclosed herein are methods for preparing articles, especially medical adhesive articles. In some embodiments, the method comprises forming a transfer tape on a release liner. This method comprises providing a release liner with a first major surface and a second major surface, wherein at least the second major surface comprises a release surface, disposing a (meth)acrylate-based pressure sensitive adhesive or a (meth)acrylate-based pressure sensitive adhesive precursor on the second major surface of the release liner and forming the (meth)acrylate-based pressure sensitive adhesive or (meth)acrylate-based pressure sensitive adhesive precursor into a (meth)acrylate-based pressure sensitive adhesive layer with a first major surface and second major surface where the first major surface of the (meth)acrylate-based pressure sensitive adhesive layer is in contact with the second major surface of the release liner. On the second major surface of the (meth)acrylate-based pressure sensitive adhesive layer is disposed a non-functional siloxane fluid to form a layer. The layer of the non-functional siloxane fluid is radiation-cured to form a siloxane gel adhesive layer with a first major surface and a second major surface, where the first major surface of the siloxane gel adhesive layer is in contact with the second major surface of the (meth)acrylate-based pressure sensitive adhesive layer. Such transfer tapes have been described in detail above and are such that the interfacial adhesion of (meth)acrylate-based pressure sensitive adhesive and the radiation-cured siloxane gel adhesive layer is sufficiently strong that the radiation-cured siloxane gel adhesive layer is not peelable from the (meth)acrylate-based pressure sensitive adhesive layer. Suitable release liners, (meth)acrylate-based pressure sensitive adhesives, and non-functional siloxane fluids are described above.

As mentioned above, the radiation-curing can be carried out in a variety of ways. Typically, the radiation-curing of the non-functional siloxane fluid comprises E-beam curing. A variety of procedures for E-beam curing are well-known. The cure depends on the specific equipment used, and those skilled in the art can define a dose calibration model for the specific equipment, geometry, and line speed, as well as other well understood process parameters.

Commercially available electron beam generating equipment is readily available, such as a Model CB-300 electron beam generating apparatus (available from Energy Sciences, Inc. (Wilmington, MA). Generally, a support film (e.g., polyester terephthalate support film) runs through a chamber. In some embodiments, the sample of uncured material on the release liner may be attached to the support film and conveyed at a fixed speed through the chamber. Generally, the chamber is inerted (e.g., the oxygen-containing room air is replaced with an inert gas, e.g., nitrogen) while the samples are e-beam cured.

In some embodiments, forming the (meth)acrylate pressure sensitive adhesive layer comprises cooling, drying, curing, crosslinking, or a combination thereof. The (meth)acrylate pressure sensitive adhesive layer can be continuous or discontinuous.

In some embodiments, disposing the non-functional siloxane fluid on the second major surface of the (meth)acrylate-based pressure sensitive adhesive layer comprises disposing the non-functional siloxane fluid in a discontinuous manor such that upon radiation-curing the siloxane gel adhesive layer is a discontinuous layer. The discontinuous layer of siloxane gel adhesive can be arrayed in a regular or a non-regular pattern. In some embodiments, the non-functional siloxane fluid is disposed on the second major surface of the (meth)acrylate-based pressure sensitive adhesive layer in a discontinuous manor by printing of the non-functional siloxane fluid.

A wide variety of patterns and shapes are suitable. The structures may have a wide variety of shapes and sizes. Representative examples of shapes include hemispheres, prisms (such as square prisms, rectangular prisms, cylindrical prisms and other similar polygonal features), pyramids, ellipses, grooves (e.g., V-grooves), channels, and the like. The particular dimensions and patterns characterizing the patterns are selected based upon the specific application for which the article is intended.

As described above, in some embodiments, the non-functional siloxane fluid is described by Formula 1:

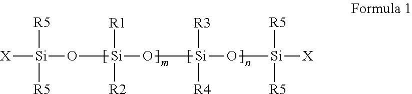

where R1, R2, R3, and R4 are independently selected from the group consisting of a substituted or unsubstituted alkyl or aryl group; each R5 is an alkyl group, each X is an R5 group or a —OH group; and n and m are integers, wherein at least one of m or n is not zero.

Also disclosed herein are articles, especially medical articles. In some embodiments, the article comprises a substrate that is not a release liner with a first major surface and a second major surface, and a transfer tape laminated to the second major surface of the substrate. Suitable transfer tapes have been described in detail above and comprise a (meth)acrylate-based pressure sensitive adhesive layer and a radiation-cured siloxane gel adhesive layer in contact with the (meth)acrylate-based pressure sensitive adhesive layer.

In some embodiments, the substrate comprises a tape backing, a film, a foam, a non-woven, a woven, an elastic cloth, an adhesive layer, a paper, or a medical device. In some embodiments, the article comprises a wound dressing, a medical drape, a medical bandage, a wound cover, a negative pressure wound therapy article, or a wearable medical construction.

An example of an article of this disclosure is shown in the Figure. The Transfer Tape 1 comprises siloxane gel adhesive layer 2, (meth)acrylate-based adhesive layer 3, and release liner 4. Release liner 4 has first major surface 5 and second major surface 6. The (meth)acrylate-based adhesive layer has first major surface 7 and second major surface 8 and is disposed on second major surface 6 of release liner 4 such that first major surface 7 of the (meth)acrylate-based adhesive layer 3 is in contact with the second major surface 6 of release liner 4. The siloxane gel adhesive layer has first major surface 9 and is disposed on second major surface 8 of the (meth)acrylate-based adhesive layer 3 such that the first major surface 9 is in contact with the second major surface 8 of the (meth)acrylate-based adhesive layer 3.

### Examples

These examples are merely for illustrative purposes only and are not meant to be limiting on the scope of the appended claims. The following abbreviations are used: mm=millimeters; cm=centimeters; dm=decimeters; m=meters; in =inches; g=grams; mJ=milliJoules; N=Newtons; kGy=kiloGrays; kV=kiloVolts; rpm=revolutions per minute. The terms “weight %”, “% by weight”, and “wt %” are used interchangeably.

Table of Abbreviations

| Abbreviation | |
|---|---|
| or Trade | |
| Designation | Description |
| Acrylate PSA-1 | Hot-melt Acrylic adhesive 2EHA/AA/A/ABP prepared as described in |
| | US Patent Publication No. 2008/0233348, Example 12 |
| 2EHA | 2-ethylhexyl acrylate |
| AA | Acrylic acid |
| Silicone Fluid-1 | Silicone Fluid available from Dow as Xiameter OHX-4070 |
| Silicone Fluid-2 | Silicone fluid available from Wacker as AK1000000 |
| Tackifier | Silicone MQ Tackifying Resin, MQ TMS803 TF available from |
| | Wacker |
| Silicone | A 2 part thermal curing silicone gel commercially available from |
| Adhesive-1 | Wacker as SILGEL 612 |
| Liner-1 | Release liner for acrylate PSAs, 1-side siliconized 100 gsm white |
| | Glassine liner (Mondi) |
| Liner-2 | Release liner for silicone PSAs, available from Siliconature as SILFUL |
| | S 50 M 1R82001 CLEAR 305 mm |
| Backing-1 | A nonwoven backing with film, as disclosed in US Patent Publication |
| | No. 2022/0127505 |
| LDPE | Low density polyethylene substrate2″ × 5″ × 3/16″ (50 × 127 × 4 mm), |
| | available from Aeromat Plastics |
| Film-1 | Film substrate of Sontara #8010 available from Jacob-Holm |
| Carrier layer | Both side PP coated paper, Aqualix PCK Y05501 from Felix-Schoeller |
| TPU | Thermoplastic polyurethane resin available as PELLETHANE 5863- |
| | 85A from Lubrizol |
| Silicone | The silicone adhesive pattern was created as described in PCT |
| Adhesive | Publication WO 2021/090113. The pattern has oval of adhesive, with |
| Pattern | individual oval size is about 3 mm × 7 mm, and about 16% of the area is |
| | silicone adhesive free area. |

### Test Methods

### Adhesion Testing

Adhesion of tape samples to a variety of substrates was tested using the test method ASTM D330 using a Zwick tensile tester machine. Measurements were made in N/in and converted to N/dm.
Substrates:
LDPE: LDPE test plates obtained from Rochol GmbH, (Germany).
Film-1: A sample of Film-1 adhered to a stainless steel plate.
ATS: Adhesion to steel.

### Examples E1-E3 and Comparative Example CE-1

### Example 1: Preparation of Transfer Tape

### Preparation of Coated Acrylate PSA

An Acrylate PSA was extruded and coated onto Liner-1 at an adhesive coating weight or thickness as shown in Table 1. The coated acrylate PSA was exposed to UV-C light with a strength of 40-60 mJ/cm2.

### Preparation and Coating of Silicone Adhesive on the Acrylate PSA Layer

Silicone Silicone Fluid-2 was mixed with Tackifier (6% by weight) in a twin-screw extruder and coated onto the acrylate PSA layer. The coating was cured by exposure to E-beam radiation. The cured silicone layer was covered by Liner-2.

TABLE 1

| | Acrylate | Acrylate | | Silicone |
|---|---|---|---|---|
| Example | Liner | PSA | Silicone Adhesive | Liner |
| E1 | Liner-1 | PSA-1 | Fluid-2 with 6% tackifier | Liner-2 |
| | | 80 g/m² coat | 125 g/m² coat weight | |
| | | weight UV | UV cured 45 mJ/cm² | |
| | | cured 45 | E-beam cured 32 kGy | |
| | | mJ/cm² | (280 kV) | |

### Examples E2-E3 and Comparative Examples CE-1: Preparation of Tape Laminates

For Example CE-1, a silicone adhesive was laminated to Backing-1, for examples E2 and E3 transfer tapes were laminated to Backing-1 on the acrylate adhesive side. The adhesives are shown in Table 2.

TABLE 2

| | Acrylate | |
|---|---|---|
| Example | Adhesive | Silicone Adhesive |
| CE1 | None | 100 micrometer thick layer of Silicone |
| | | Fluid-1 with 8% Tackifier; |
| | | E-beam Cured 60 kGy (280 kV) |
| E2 | 40 micrometer | 60 micrometer thick layer of Silicone |
| | thick layer of | Fluid-1 with 8% Tackifier; |
| | Acrylate PSA-1; | E-beam Cured 60 kGy (280 kV) |
| | Cured by UV | |
| | 45 mJ/cm² | |
| E3 | 80 micrometer | 20 micrometer thick layer of Silicone |
| | thick layer of | Fluid-1 with 8% Tackifier; |
| | Acrylate PSA-1; | E-beam Cured 60 kGy (280 kV) |
| | Cured by UV | |
| | 45 mJ/cm² | |

### Tape Testing of Examples E2-E3 and CE1

### A. Adhesion Testing to LDPE

The tape samples were laminated to an LDPE substrate and tested (TO) and aged for 34 days at 150° F. (66 °C) and tested. The data are shown in Table 3.

TABLE 3
Adhesion Testing to LDPE

| | T0 | T0 | Day 34 | Day 34 |
|---|---|---|---|---|
| Example | (N/in) | (N/dm) | (N/in) | (N/dm) |
| CE-1 | 1.23 | 4.84 | 1.23 | 4.84 |
| E2 | 1.24 | 4.88 | 1.16 | 4.57 |
| E3 | 1.34 | 5.27 | 1.23 | 4.84 |

### B. Adhesion Testing to Film-1

The tape samples were laminated to a Film-1 substrate and tested (TO) and aged for 34 days at 150° F. (66 °C) and tested. The data are shown in Table 4.

TABLE 4
Adhesion Testing to Film-1

| | T0 | T0 | Day 34 | Day 34 |
|---|---|---|---|---|
| Example | (N/in) | (N/dm) | (N/in) | (N/dm) |
| CE-1 | 0.05 | 0.20 | 0.12 | 0.47 |
| E2 | 0.27 | 1.06 | 0.46 | 1.81 |
| E3 | 0.53 | 2.08 | 0.44 | 1.73 |

Adhesion to the Film-1 substrate, which has a rough surface suggests an improvement of adhesion to human skin.

### Example E4: Patterned Silicone Adhesive Transfer Tape

### Example 4: Preparation of Patterned Silicone Adhesive Transfer Tape

Preparation of coated Acrylate PSA

An Acrylate PSA was extruded and coated onto Liner-1 at an adhesive coating weight or thickness as shown in Table 5. The coated acrylate PSA was exposed to UV-C light with a strength of 45 mJ/cm2.

### Preparation and Coating of Silicone Adhesive on the Acrylate PSA Layer

Silicone Fluid-2 was mixed with Tackifier (6% by weight) in a twin-screw extruder and coated onto the acrylate PSA layer in a pattern. The coating was cured by exposure to E-beam radiation. The cured silicone layer was covered by Liner-2.

TABLE 5

| | Acrylate | Acrylate | | Silicone |
|---|---|---|---|---|
| Example | Liner | PSA | Silicone Adhesive | Liner |
| E4 | Liner-1 | PSA-2 | Fluid-2 with 6% tackifier | Liner-2 |
| | | 80 | UV cured 45 mJ/cm² | |
| | | micrometer | E-beam cured 32 kGy | |
| | | thickness | (280 kV) | |
| | | | 200 micrometer thickness | |

### Examples E5 and Comparative Example CE-2-CE-4: Tape Articles

### Example 5: Preparation of Tape

The preparation of a tape in a continuous process by forming a tape backing and coating the acrylate PSA and then a patterned silicone adhesive layer was used as shown in Table 6.

### Preparation of Backing Layer

On a moving Carrier Layer, a layer of TPU was extruded onto the Carrier layer at a coat weight of 30 g/m² to form a TPU layer.

Preparation of coated Acrylate PSA

An Acrylate PSA was extruded and coated onto the TPU layer at an adhesive coating weight or thickness of 40 g/m² to form an acrylate PSA layer. The coated acrylate PSA was exposed to UV-C light with a strength of 40 mJ/cm2.

### Preparation and Coating of Silicone Adhesive on the Acrylate PSA Layer

Silicone Fluid-2 were mixed with Tackifier (6% by weight) in a twin-screw extruder and pattern coated onto the acrylate PSA layer. The coating was cured by exposure to UV radiation followed by E-beam radiation. The cured silicone layer was covered by Liner-2.

TABLE 6

| | TPU | | | Silicone |
|---|---|---|---|---|
| Example | Layer | Acrylate PSA | Silicone Adhesive | Liner |
| E5 | 30 g/m² | PSA-1 | Fluid-2 with 6% tackifier | Liner-2 |
| | | 40 g/m² coat | 170 g/m² coat weight | |
| | | weight UV | UV cured 40 mJ/cm² | |
| | | cured 40 | E-beam cured 28 kGy | |
| | | mJ/cm² | (280 kV) | |

### Examples CE2-CE4: Preparation of Tape

For comparison a series of 2-layer tapes were prepared as described above with: CE-2 TPU backing/pattern coated silicone fluid; CE-3 TPU backing and un-patterned silicone fluid; and CE-4 TPU backing with PSA-1. The details of these tapes are shown in Table 7.

TABLE 7

| | TPU | Acrylate | | |
|---|---|---|---|---|
| Example | Layer | PSA | Silicone Adhesive | Liner |
| CE-2 | 30 | None | Patterned | Liner-2 |
| | g/m² | | Fluid-2 with 6% tackifier | |
| | | | E-beam cured 28 kGy | |
| | | | 170 g/m² | |
| CE-3 | 30 | None | Unpatterned | Liner-2 |
| | g/m² | | Fluid-2 with 6% tackifier | |
| | | | E-beam cured 30 kGy | |
| | | | 225 g/m² | |
| CE-4 | 30 | PSA-1 | None | Liner-2 |
| | g/m² | 40 g/m² | | |
| | | coat weight | | |
| | | UV cured 40 | | |
| | | mJ/cm² E-beam | | |
| | | cured 20 kGy | | |

### Examples E5 and CE2-CE4: Tape Tests

The above prepared tape samples were tested for ATS (Adhesion to Steel) The results are shown in Table 8.

TABLE 8

| Example | ATS (N/in) | ATS (N/dm) |
|---|---|---|
| E-5 | 3.61 | 14.2 |
| CE-2 | 2.58 | 10.2 |
| CE-3 | 3.39 | 13.3 |
| CE-4 | 8.44 | 33.2 |

### Preparation of Comparative Example CE5:

### Preparation of Coated Acrylate PSA

Acrylate PSA-1 was extruded and coated onto TPU at an adhesive coating weight or thickness of 40 g/m² to form an acrylate PSA layer. The coated acrylate PSA was exposed to UV-C light with a strength of 40 mJ/cm2.

### Preparation of Coated Silicone PSA

Silicone Fluid-1 was mixed with Tackifier (8% by weight) in a twin-screw extruder and coated onto Liner-2. The coating was cured by exposure to E-beam radiation at 60 kGy (280 kV).

### Lamination of Acrylate PSA and Silicone PSA

The coated Acrylate PSA and Silicone PSA samples prepared above were laminated using a nip press lamination process.

### Preparation of Comparative Example CE-6:

### Preparation of Coated Acrylate PSA

Acrylate PSA-1 was extruded and coated onto Liner-1 at an adhesive coating weight or thickness as shown in Table 1. The coated acrylate PSA was exposed to UV-C light with a strength of 40-60 mJ/cm2.

### Preparation and Coating of Silicone Gel on the Acrylate PSA Layer

A 1:1 mixture of Part A and Part B of Silicone Adhesive-1 was weighed into a jar, mixed in a SpeedMixer (Hausschild GmbH, Germany) for 30 seconds at 1000 rpm. The homogeneous mixture was coated onto the acrylic PSA using a suitable Meyer Bar to achieve a wet coating thickness of 100 micrometers. The construction was heat-cured in an oven at 120 °C for 10 min and covered with Liner-2. After 72 hours, Liner-2 was released prior to testing.

### Example E-3, CE-5, CE-6: Testing of Cohesive Strength

Samples were tested for cohesive strength of the two adhesive layers (acrylate and silicone) by pulling apart the layers by hand. Cohesive fail is defined as separation of silicone and acrylate layer upon pull. The results are presented in Table 9. This test demonstrates that laminating together the cured acrylate and silicone adhesive layers, or thermally curing a silicone adhesive on a coating of acrylate adhesive does not produce the same desirable high cohesive strength between the adhesive layers as the exemplary adhesive layers.

TABLE 9

| Example | Cohesive Fail |
|---|---|
| E-3 | No |
| CE-5 | Yes |
| CE-6 | Yes |

1. A transfer tape, comprising:
a release liner with a first major surface and a second major surface, wherein at least the second major surface is a release surface;
a (meth)acrylate-based pressure sensitive adhesive layer with a first major surface and a second major surface, wherein the first major surface is in contact with the second major surface of the release liner, and wherein the (meth)acrylate-based pressure sensitive adhesive layer is a continuous layer; and
a radiation-cured siloxane gel adhesive layer with a first major surface and a second major surface, wherein the first major surface of the siloxane gel adhesive layer is in contact with the second major surface of the (meth)acrylate-based pressure sensitive adhesive layer, and wherein the radiation-cured siloxane gel adhesive layer is formed by radiation curing of a non-functional siloxane fluid, wherein the non-functional siloxane fluid is in contact with the (meth)acrylate-based pressure sensitive adhesive layer when cured, such that the interfacial adhesion of (meth)acrylate-based pressure sensitive adhesive and the radiation-cured siloxane gel adhesive layer is sufficiently strong that the radiation-cured siloxane gel adhesive layer is not peelable from the (meth)acrylate-based pressure sensitive adhesive layer.

2. The transfer tape of claim 1, wherein the radiation curing comprises E-beam curing.

3. The transfer tape of claim 1, wherein the non-functional siloxane fluid is described by Formula 1: 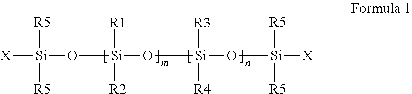
wherein R1, R2, R3, and R4 are independently selected from the group consisting of a substituted or unsubstituted alkyl or aryl group;
each R5 is an alkyl group, each X is an R5 group or a —OH group; and
n and m are integers, wherein at least one of m or n is not zero.

4. The transfer tape of claim 1, wherein the (meth)acrylate-based pressure sensitive adhesive layer has a thickness of from 10-100 micrometers and the radiation-cured siloxane gel adhesive layer has a thickness of from 25-500 micrometers

5. The transfer tape of claim 1, wherein the radiation-cured siloxane gel adhesive layer is a discontinuous layer.

6. (canceled)

7. The transfer tape of claim 5, wherein the discontinuous radiation-cured siloxane gel adhesive layer is formed by forming a discontinuous layer of non-functional siloxane fluid.

8. The transfer tape of claim 7, wherein the discontinuous layer of non-functional siloxane fluid is formed by printing.

9. A method for preparing an article, the method comprising:
forming a transfer tape on a release liner, wherein forming the transfer tape comprises:
providing a release liner with a first major surface and a second major surface, wherein at least the second major surface comprises a release surface;
disposing a (meth)acrylate-based pressure sensitive adhesive or a (meth)acrylate-based pressure sensitive adhesive precursor on the second major surface of the release liner; and
forming the (meth)acrylate-based pressure sensitive adhesive or (meth)acrylate-based pressure sensitive adhesive precursor into a (meth)acrylate-based pressure sensitive adhesive layer with a first major surface and second major surface, wherein the first major surface of the (meth)acrylate-based pressure sensitive adhesive layer is in contact with the second major surface of the release liner;
disposing a non-functional siloxane fluid on the second major surface of the (meth)acrylate-based pressure sensitive adhesive layer; and
radiation-curing of the non-functional siloxane fluid to form a siloxane gel adhesive layer with a first major surface and a second major surface, wherein the first major surface of the siloxane gel adhesive layer is in contact with the second major surface of the (meth)acrylate-based pressure sensitive adhesive layer, such that the interfacial adhesion of (meth)acrylate-based pressure sensitive adhesive and the radiation-cured siloxane gel adhesive layer is sufficiently strong that the radiation-cured siloxane gel adhesive layer is not peelable from the (meth)acrylate-based pressure sensitive adhesive layer.

10. The method of claim 9, wherein radiation-curing of the non-functional siloxane fluid comprises E-beam curing.

11. The method of claim 9, wherein forming the (meth)acrylate pressure sensitive adhesive layer comprises forming a continuous layer or discontinuous layer and comprises cooling, drying, curing, crosslinking, or a combination thereof.

12. The method of claim 11, wherein the (meth)acrylate pressure sensitive adhesive layer comprises a discontinuous layer.

13. The method of claim 9, further comprising:
peeling the transfer tape from the surface of the release liner;
providing a substrate that is not a release liner with a first major surface and a second major surface; and
laminating the (meth)acrylate-based pressure sensitive adhesive layer surface of the transfer tape to second major surface of the substrate.

14. The method of claim 13, wherein the substrate comprises a tape backing, a film, a foam, a non-woven, a woven, an elastic cloth, an adhesive layer, a paper, or a medical device.

15. The method of claim 9, wherein disposing the non-functional siloxane fluid on the second major surface of the (meth)acrylate-based pressure sensitive adhesive layer comprises disposing the non-functional siloxane fluid in a discontinuous manor such that upon radiation-curing the siloxane gel adhesive layer is a discontinuous layer.

16. The method of claim 15, wherein the discontinuous layer of siloxane gel adhesive is arrayed in a regular or a non-regular pattern.

17. The method of claim 15, wherein the non-functional siloxane fluid is disposed on the second major surface of the (meth)acrylate-based pressure sensitive adhesive layer in a discontinuous manor by printing of the non-functional siloxane fluid.

18. The method of claim 9, wherein the non-functional siloxane fluid is described by Formula 1: 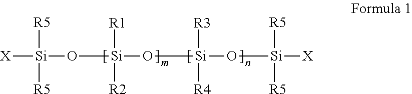
wherein R1, R2, R3, and R4 are independently selected from the group consisting of a substituted or unsubstituted alkyl or aryl group;
each R5 is an alkyl group, each X is an R5 group or a —OH group; and
n and m are integers, wherein at least one of m or n is not zero.

19. A medical article comprising:
a substrate that is not a release liner with a first major surface and a second major surface; and
a multi-layer adhesive laminated to the second major surface of the substrate, wherein the multi-layer adhesive comprises:
a (meth)acrylate-based pressure sensitive adhesive layer with a first major surface and a second major surface, wherein the first major surface is in contact with the second major surface of the release liner; and
a radiation-cured siloxane gel adhesive layer with a first major surface and a second major surface wherein the first major surface of the siloxane gel adhesive layer is in contact with the second major surface of the (meth)acrylate-based pressure sensitive adhesive layer, and wherein the radiation-cured siloxane gel adhesive layer is formed by radiation-curing of a non-functional siloxane fluid wherein the non-functional siloxane fluid is in contact with the (meth)acrylate-based pressure sensitive adhesive layer when cured such that the interfacial adhesion of (meth)acrylate-based pressure sensitive adhesive and the radiation-cured siloxane gel adhesive layer is sufficiently strong that the radiation-cured siloxane gel adhesive layer is not peelable from the (meth)acrylate-based pressure sensitive adhesive layer.

20. The medical article of claim 19, wherein the substrate comprises a tape backing, a film, a foam, a non-woven, a woven, an elastic cloth, an adhesive layer, a paper, or a medical device.

21. The medical article of claim 19, wherein the article comprises a wound dressing, a medical drape, a medical bandage, a wound cover, a negative pressure wound therapy article, or a wearable medical construction.
